(19) United States
(12) Patent Application Publication
(10) Pub. No.: US 2023/0310461 A1
(43) Pub. Date: Oct. 5, 2023

(54) Compositions and Methods for Treatment of Prediabetes

(71) Applicant: William H. Cross, III, Waco, GA (US)

(72) Inventor: William H. Cross, III, Waco, GA (US)

(21) Appl. No.: 18/330,102

(22) Filed: Jun. 6, 2023

Related U.S. Application Data
(63) Continuation-in-part of application No. 15/650,825, filed on Jul. 14, 2017, now abandoned.
Application No. 15/650,825 is a continuation-in-part of application No. 15/496,919, filed on Apr. 25, 2017, now Pat. No. 11,612,632.
(60) Provisional application No. 62/327,421, filed on Apr. 25, 2016.

(51) Int. Cl. A61K 31/593; A61K 31/352; A61K 9/00; A61K 31/385; A61K 31/714; A61K 36/87; A61K 31/198; A61K 31/205; A61K 31/4172; A61K 31/155; A61K 31/047

(52) U.S. Cl. CPC A61K 31/593; A61K 31/352; A61K 9/0053; A61K 31/385; A61K 31/714; A61K 36/87; A61K 31/198; A61K 31/205; A61K 31/4172; A61K 31/155; A61K 31/047

(57) ABSTRACT

Described herein are compositions and methods to treat and prevent development of prediabetes. In particular, the present invention has discovered that combining certain diverse types of antioxidants yields synergistic and surprising effects against prediabetes. These antioxidant types include: heteroatom-based antioxidants from a particular chemical structural antioxidants with a conjugated segment of alternating single and double bonds; antioxidants with a disulfide bond; and antioxidants having a flavan-3-ol skeleton. It has further been discovered that it is beneficial to include one or more antioxidants that facilitates an oxidative balance.

### CROSS-REFERENCE TO RELATED APPLICATIONS

This application is a continuation in part of nonprovisional U.S. patent application Ser. No. 15/650,825, filed Jul. 14, 2017, which is a continuation in part of nonprovisional U.S. patent application Ser. No. 15/496,919, filed Apr. 25, 2017, which claims priority upon provisional U.S. patent application No. 62/327,421, filed Apr. 25, 2016. Each reference is incorporated by reference in its entirety.

### BACKGROUND

Prediabetes—formerly known as borderline diabetes—is a metabolic condition affecting 86 million Americans and characterized by blood sugar levels that are higher than the normal range yet below those of diabetes mellitus. Prediabetic patients lack one or more symptoms that characterize diabetes, but the condition is often a prelude to type 2 diabetes mellitus (T2DM). A quarter of the cases commonly progress to T2DM within 3 to 5 years, and half within 10 years. Prediabetes also often has a role in heart disease and stroke. Prediabetes sometimes may be identical to metabolic syndrome, but the latter is evaluated by a different set of biomarkers.

A common symptom of prediabetes is impaired fasting glucose (IFG), in which blood sugar levels are high but still below those typical of diabetes. In IFG the fasting blood glucose or the 3-month average blood glucose (A1C) is elevated above normal levels but not extreme. It is associated with insulin resistance and an increased risk of heart disease and mortality.

Some IFG patients respond normally to a glucose tolerance test, whereas others have impaired glucose tolerance (IGT). IGT may precede T2DM by many years, and like IFG it is associated with insulin resistance. IGT is a more reliable indicator than IFG for increased risk of heart disease and mortality. Among American adults over 40 years of age, 33% have IFG, 15% have IGT, and 40% are prediabetic (i.e., they have IFG and/or IGT). Children have similar risks.

Not all cases of prediabetes can be diagnosed by IFG or IGT alone, so a range of symptoms are monitored: persistent hunger; obesity, especially abdominal or visceral obesity, and especially a body mass index over 25; weight gain or at the other extreme, unexplained weight loss; flu-like weakness and fatigue; blurry vision; slowness of healing for cuts and bruises; tingling or insensate extremities; frequency of urination; and recurring infections of the skin, gums, bladder or vagina.

The diagnostic tests include the fasting plasma glucose (FPG) test: prediabetic blood sugar is in the range of 100-125 mg/dL, equivalent to 5.6 mM/L to 6.9 mM/L (numbers above those indicate diabetes). The oral glucose tolerance test (OGTT) follows immediately after the fasting test just described and administers 75 grams of a sugary solution. Two hours later the blood sugar is tested again: 140-199 mg/dL indicates prediabetes (or diabetes if the number is higher). The hemoglobin A1C test, also known as the average blood sugar test, HbA1c test or glycohemoglobin test, determines average percentage of hemoglobin in the blood that is attached to glucose over the recent two to three months: 5.7% to 6.4% is diagnostic for prediabetes; above that indicates diabetes (or uncontrolled diabetes if it is being treated).

Prediabetes is associated with many conditions, including sleep disorders, genetics, cardiovascular disease, hypertension, high triglyceride levels, low HDL cholesterol, elevated weight, pregnancy and gestational diabetes, high birth weight, polycystic ovarian syndrome, and race (especially for African-Americans, Hispanic Americans, Native Americans, and Asian Americans). Among the lifestyle intervention guidelines to prevent the onset of T2DM are: dietary regimes that are low in sugar, refined carbohydrates, saturated fats, salt and total calories; regular physical exercise, for instance 30 minutes per day five days per week; and weight reduction even by just a few percent. Such measures can also cure prediabetes in at least some cases, though more exercise is recommended, such as 45 minutes per day.

Early intervention is urgent, both because diagnosis often misses prediabetes, and because the type of damage caused by T2DM may already be starting even in the prediabetic stage. Diabetic types of effects include atherosclerosis within the cardiovascular larger arteries. Other effects occur at the small blood vessels, with particular damage noted in the retinas, the kidneys, and the nerves. Currently metformin is the only drug recommended by the American Diabetes Association to prevent the onset of T2DM in prediabetic patients.

The incidence of prediabetes remains high despite widespread monitoring and health education. And methods to treat it have been lacking apart from the lifestyle changes just discussed. Thus there is an ongoing need for compositions to treat and prevent prediabetes.

### SUMMARY

Described herein are compositions and methods to treat and prevent development of prediabetes. In particular, the present invention has discovered that combining certain diverse types of antioxidants yields synergistic and surprising effects against prediabetes. These antioxidant types include: heteroatom-based antioxidants from a particular chemical structural antioxidants with a conjugated segment of alternating single and double bonds; antioxidants with a disulfide bond; and antioxidants having a flavan-3-ol skeleton. It has further been discovered that it is beneficial to include one or more antioxidants that facilitates an oxidative balance.

In one aspect, unit dose composition comprises the following antioxidant compounds:
a) a plurality of heteroatom-containing natural antioxidant compounds have the formula X—CH(R¹)—CH(R²)—(CH₂)ₘ—Z, wherein:
   i) X is one of the following moieties or a pharmaceutically acceptable salt thereof: —NH₂ or —N(CH₃)₃;
   ii) R¹ is —H, —CO2H, or a pharmaceutically acceptable salt of —CO2H;
   iii) R² is —H, —OH, or —O-acetyl;
   iv) m is 0, 1, 2, or 3; and
   v) Z is one of the following moieties or a pharmaceutically acceptable salt thereof: —SH, —SO₂H, —SO₃H, —CO₂H, —N═C(—NH₂)—NH₂, —CH(—NH₂)—CO₂H; -imidazolyl; or —C(═O)—NH—CH(—CO₂H)—CH₂—Y where Y is 4-imidazolyl or 1-methyl-4-imidazolyl;
b) a plurality of antioxidant compounds have a conjugated segment, comprising at least one vitamin D compound and at least one cobalamin compound;
c) at least one of the antioxidant compounds comprises a disulfide bond; and
d) at least one of the antioxidant compounds is a flavan-3-ol compound.

In another aspect, described herein are methods for the treatment of prediabetes comprising administering a unit dose composition described herein to the subject.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient” include, but are not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “composition” as used with reference to compositions of the invention means a food supplement composition or pharmaceutical composition.

The term “prediabetes” means a metabolic condition identified by at least one of the following: fasting plasma glucose level in the range of 100-125 mg/dL; blood sugar in an oral glucose tolerance test in the range of 140-199 mg/dL; or hemoglobin A1C in the range of 5.7% to 6.4% in a hemoglobin A1C test.

The terms “diabetes” means a metabolic condition identified by at least one of the following: fasting plasma glucose level >126 mg/dL; blood sugar in an oral glucose tolerance test >200 mg/dL; or hemoglobin A1C >6.5% in a hemoglobin A1C test.

The term “symptom of prediabetes” means a symptom that is commonly used by the allopathic medical community to monitor or diagnose prediabetes. Examples of symptoms of prediabetes include: IFG; IGT; persistent hunger; obesity; abdominal obesity; visceral obesity; a body mass index over 25; weight gain; unexplained weight loss; flu-like weakness; fatigue; blurry vision; slowness of healing for cuts; slowness of healing for bruises; a tingling extremity; an insensate extremity; frequent need to urinate; a recurring infection of the skin; a recurring infection of the gums; a recurring infection of the bladder; a recurring infection of the vagina; blood sugar in the range of 100-125 mg/dL or 5.6 mM/L to 6.9 mM/L for the fasting plasma glucose test; blood sugar in the range of 140-199 mg/dL for the oral glucose tolerance test; or average hemoglobin A1C of 5.7% to 6.4% in the hemoglobin A1C test.

The term “reduce or mitigate” as used with respect to a symptom of prediabetes means to decrease the manifestation of the symptom by an amount that is customarily considered medically significant, measurable and relevant. In a particular embodiment the reduction or mitigation decreases the deviation from a healthy normal range by at least 10%.

The terms “method” as used with respect to treatment of prediabetes or its symptoms according to the invention means a protocol to reduce or mitigate the symptoms thereof by providing a composition of the invention. The term “treatment” as used with respect to prediabetes means use of such a method.

The term “administration of a composition” means to provide a dose of the composition for ingestion by someone in need thereof.

The term “antioxidant” as used with respect to a compound means that the molecule can inhibit oxidation or reactions promoted by oxygen, peroxides, or free radicals. Important examples of antioxidant compounds include vitamin C, vitamin E, phenols, and zinc. Various types of antioxidant activity exist. Some antioxidants such as zinc donate an electron to reduce a free radical or oxidizing species. Some antioxidants bond to a free radical species to terminate a radical chain reaction. Some antioxidants such as phenols donate a hydrogen atom to a free radical species to terminate a radical chain reaction and form a stabilized radical that then reacts by another type of reaction to eliminate the radical.

The term “pro-oxidant” as used with respect to a compound means that the molecule can promote oxidation or other reactions that are commonly mediated by oxygen, peroxides, or free radicals. This property is counterintuitive for antioxidants but several examples are known, particularly for vitamins C and E. Thus for instance ascorbate (vitamin C) reduces Fe(III) to produce Fe(II), which can then react with hydrogen peroxide (an oxidizing substance) by the Fenton reaction to generate hydroxyl free radicals by the following redox cycling reactions: 2 Fe²⁺+2H₂O₂→2 Fe³⁺+2 OH·+2 OH⁻; 2 Fe³⁺+Ascorbate→2 Fe²⁺+Dehydroascorbate. Thus antioxidants that can serve as reduction agents may have a pro-oxidant effect at suitable concentrations and if O₂ or peroxide and a transition metal are present.

The terms “pro-oxidative effect” and “pro-oxidant activity” as used with respect to a compound's effect are used synonymously and mean that the molecule acts as a pro-oxidant in that environment. Pro-oxidative effects may be associated with the generation of oxidative stress in tissues, however in some cases that stress may mimic a normal physiological signal, for instance for the conversion of fibroblasts to myofibroblasts in fibrosis or scar formation. Where an antioxidant has a pro-oxidative effect, the pro-oxidative effect may be different in mechanism and strength from that of the antioxidant activity of the molecule.

The term “pro-oxidative effect in the liver” as used with respect to a compound means that the molecule has pro-oxidant activity that is known to occur at least in the liver. In particularly useful embodiments of the invention one or more of the antioxidants that comprise stabilizing heteroatoms have pro-oxidant activity in the liver.

The term “type” as used with respect to an antioxidant compound means its structural category. As defined herein, types of antioxidant compound include at least: heteroatom-containing natural antioxidant compounds have the formula X—CH(R¹)—CH(R²)—(CH₂)ₘ—Z; those comprising a conjugated segment; those comprising a disulfide bond; and those having a flavan-3-ol skeleton. Typically there is no overlap between these categories, however the invention is not so limited.

The term “heteroatom-containing natural antioxidant compound” means a natural compound that is an antioxidant and that contains a heteroatom. As used herein this description is coupled with a generic chemical formula X—CH(R¹)—CH(R²)—(CH₂)ₘ—Z, wherein:
i) X is one of the following moieties or a pharmaceutically acceptable salt thereof: —NH₂ or —N(CH₃)₃;
ii) R¹ is —H, —CO₂H, or a pharmaceutically acceptable salt of —CO₂H;
iii) R² is —H, —OH, or —O-acetyl;
iv) m is 0, 1, 2, or 3; and
v) Z is one of the following moieties or a pharmaceutically acceptable salt thereof: —SH, —SO₂H, —SO₃H, —CO₂H, —N—C(—NH₂)—NH₂, —CH(—NH₂)—CO₂H; -imidazolyl; or —C(═O)—NH—CH(—CO₂H)—CH₂—Y where Y is 4-imidazolyl or 1-methyl-4-imidazolyl.
   The chemical notations, terminology, and structural designations there follow the conventions of the art of chemistry, except that the identities X, Y, and Z are understood to correspond to moieties that are specific to the individual compound but vary across the category.

The term “heteroatom” means any atom in an organic molecule that is not carbon or hydrogen, where the term “organic” as used with respect to a molecule has its usual and ordinary meaning in organic chemistry. Illustrative examples of heteroatoms include but are not limited to nitrogen, oxygen, phosphorus, sulfur, selenium, boron, fluorine, chlorine, bromine, and iodine, as those atoms are named on the periodic table.

The term “stabilizing heteroatom” as used with respect to an antioxidant compound means a heteroatom that is capable of donating electron density toward a radical on a neighboring carbon atom in a molecule, and or that possesses an empty orbital to accept electron density from such a radical. Examples of stabilizing heteroatoms include boron, nitrogen, oxygen, phosphorus and sulfur but the invention is not so limited. Preferred stabilizing heteroatoms are nitrogen, oxygen and sulfur, and a particularly preferred stabilizing heteroatom is sulfur but the invention is not so limited.

The term “saturated carbon” as used with respect to an atom in a compound means that it is a carbon atom for which bonds to its neighboring atoms are all and only single bonds, wherein the recited carbon atom is bonded to at least one other carbon atom and to at least one hydrogen atom. As used herein, the term “saturated carbon” contemplates that the carbon atom may be covalently bonded to one or more heteroatoms, but does not necessarily have such a bond. The term “adjacent to” as used with respect to one atom and another in a compound means that the two are covalently bonded to one another.

The terms “taurine”, “beta-alanine”, “acetyl-carnitine”, “agmatine”, “carnosine” “cysteine”, “sulfinoalanine”, “hypotaurine”, “carnitine”, “lysine”, “anserine”, “histidine”, “arginine”, and “ornithine” have their usual and ordinary meanings in the fields of chemistry and biochemistry.

The term “conjugated segment” as used with respect to a molecule means a portion of the molecular skeleton that has single bonds alternating with multiple bonds such as double or triple bonds. In particular embodiments the skeleton of the conjugated segment is composed entirely of carbon atoms. In further embodiments the skeleton of the conjugated segment is composed of carbon atoms and one or more heteroatoms. In preferred embodiments the conjugated segment is covalently bonded to at least one saturated carbon atom such as a methyl group (i.e. —CH₃), a substituted methyl group (i.e., —CH₂X or —CHXY where X and Y are, e.g., halogen atoms that may be the same or different), a methylene group (i.e., —CH₂—), a singly substituted methylene group (i.e., —CHX—, where X is, e.g., a halogen atom), or a tertiary carbon that is bonded to hydrogen. Non-limiting illustrative examples of conjugated segments in compounds that are suitable for use with the invention include: the porphyrins rings of cobalamin compounds; the conjugated portions of rings, branches and linear backbone in vitamin D compounds such as ergocalciferol (vitamin D₂) and cholecalciferol (vitamin D₃); and the linear chains of the carotenoids, as well as cyclic end groups of the carotenoids to the extent that they possess a C═C double bond conjugated with the linear chain.

The term “vitamin D compound” means a molecule that is within the group known as vitamin D. These are secosteroids and in particular include the following: 7-dehydrocholesterol, also known as previtamin-D₃; vitamin D₂, also known as ergocholecalciferol; vitamin D₃, also known as cholecalciferol and activated 7-dehydrocholesterol; 25-D, also known as 25-hydroxyvitamin D, calcidiol, and calcifediol; and 1,25-D, also known as 1,25-dihydroxycholecalciferol, 1,25-dihydroxyvitamin D₃, and calcitriol.

The term “cobalamin compound” means a compounds having a skeleton of cobalamin (i.e., vitamin B₁₂, i.e., α-(5,6-dimethylbenzimidazolyl)cobalamide) with an axial ligand on the cobalt atom, where the ligand is not bridged to the rest of the molecule. In nonlimiting illustrative examples that axial ligand is: —CH₃ for methylcobalamin; —OH for hydroxocobalamin; —CN for cyanocobalamin; and -Ado for adenosylcobalamin, where -Ado is an adenosyl moiety. In a particular embodiment of the invention methylcobalamin is the form of cobalamin used, but the invention is not so limited. As used herein, the term “vitamin B₁₂” is used synonymously with cobalamin, and includes all active forms of cobalamin. When a weight quantity of cobalamin or vitamin B₁₂ is given herein, it will be understood to refer to the weight quantity of the particular cobalamin species under consideration, including the R-group, regardless of which R-group is appended.

The terms “vitamin D₃” and “methylcobalamin” have their usual and ordinary meanings in the fields of chemistry and biochemistry.

The term “carotenoid” has its usual and ordinary meaning in natural products chemistry.

The terms “carotene”, “lycopene”, “neurosporene”, “phytofluene”, “phytoene”, “xanthophyll”, “canthaxanthin”, “cryptoxanthin”, “zeaxanthin”, “astaxanthin”, “lutein”, and “rubixanthin” have their usual and ordinary meaning in the fields of chemistry and natural products.

The term “lettered carotenes” means carotene compounds designated by Greek letters and for example includes alpha-carotene, beta-carotene, gamma-carotene, delta-carotene, epsilon-carotene and zeta-carotene.

The term “disulfide bond” as used with respect to a molecule means a single covalent bond that links two neighboring divalent sulfur atoms, forming an —S—S— moiety in a molecule. As used herein with respect to a sulfur atom, the phrase “is not part of a disulfide bond” means that the atom is not part of an —S—S— moiety, whereas a sulfur atom that is part of a disulfide bond is part of an —S—S— moiety.

The terms “alpha-lipoic acid”, “lipoamide”, “glutathione disulfide”, “cystine”, “asparagusic acid”, “lenthionine”, “ajoene”, “allithiamine”, “allyl propyl disulfide”, “diallyl disulfide”, “fursultiamine”, “pantethine”, “prosultiamine”, “pyritinol”, and “sulbutiamine” have their usual and ordinary meanings in the fields of chemistry and biochemistry.

The term “phenolic group” as used with respect to a molecule means a benzene ring that bears at least one hydroxy (—OH) substituent. In certain embodiments the benzene ring may be a fused ring, for instance such as is found in flavan-3-ols. In certain embodiment the phenolic group may be part of a condensed phenolic system such as tannic acid or ellagic acid. Non-limiting illustrative examples of phenolic substituents that may be found in ingredients according to the invention include those bonded to the ring or by an ether or ester of phenol (hydroxybenzene), pyrocatechol (o-dihydroxybenzene), resorcinol (m-dihydroxybenzene), pyrogallol (1,2,3-trihydroxybenzene), or phloroglucinol (1,3,5-trihydroxybenzene). In some embodiments the phenol has a C—C or C—O bond to a sugar moiety.

The term “flavan-3-ol compound” means a natural derivative of flavan-3-ol, i.e., it has moieties bonded to the 2-phenyl-3,4-dihydro-2H-chromen-3-ol skeleton shown below.

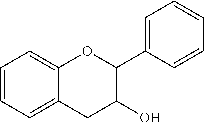 [SMILES: OC1Cc2ccccc2OC1c1ccccc1]

The moieties are typically hydroxyl groups on the aromatic rings, but may include covalent bonds that form an oligomer or polymer, and in some cases may include sugar groups as in a glycoside. The term flavan-3-ol compound as used herein includes but is not limited to catechin (C), epicatechin (EC), gallate (G), epigallocatechin (EGC), epigallocatechin gallate (EGCG), theaflavins, theaflavin-3-gallate, thearubigins, and stereoisomers and glycosides of those compounds. The term flavan-3-ol compound as used herein further includes oligomers and polymers of those compounds, in particular the proanthocyanidins and procyanidins. The terms “tannin”, “condensed tannin”, “hydrolysable tannin”, “proanthocyanidin”, “procyanidin”, “prodelphinidin”, “proguibourtinidin”, and “prorobinetinidin” as used herein have their usual and ordinary meanings in natural products and plant biochemistry, and are for instance consistent with the respective usage of such terms in CHEMISTRY AND SIGNIFICANCE OF CONDENSED TANNINS, Richard W. Hemingway and Joseph J. Karchesy, eds. (1989).

The term “glycoside” as used with respect to a compound or moiety means that it has a sugar residue covalently bonded thereto; the sugar may be a monosaccharide, disaccharide, trisaccharide, or higher order saccharide.

The terms “catechin”, “epicatechin”, “epigallocatechin”, “epicatechin gallate”, “epigallocatechin gallate”, “epiafzelechin”, “fisetinidol”, “guibourtinidol”, “mesquitol”, and robinetinidol” refer to flavan-3-ol compounds and have their usual and ordinary meaning in the fields of organic chemistry and natural products, and include the various stereoisomers thereof. The generic chemical structure for catechins and epicatechins is shown below. Where the C-2 and C-3 carbons define a trans configuration the molecule is a catechin. Where they define a cis configuration the molecule is an epicatechin.

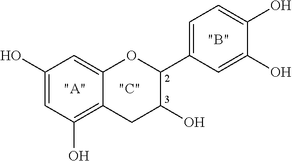 [SMILES: Oc1cc(O)c2c(c1)OC(c1ccc(O)c(O)c1)C(O)C2]

The term “derivative” as used with respect to a molecule means a variant in which the molecule is bonded to another chemical moiety.

The terms “monomer”, “oligomer”, and “polymer” as used herein with respect to a structural unit have their usual and ordinary meaning in the arts of chemistry, biochemistry and polymer science, where a monomer has one unit, an oligomer has at least two such units covalently bonded to one another, and a polymer has many such units. As an example, typically procyanidins are concatenated oligomers of catechins and or epicatechins, having from 2 to 50 or more structural units in a linear molecular chain. As a further example, proanthocyanidins, of which procyanidins are one subset, may further include gallate units, and their oligomers and polymers may be either linear or branched or in some cases cross-linked.

The term “extract” as used with respect to a botanical substance means a product that has been obtained by removal from a plant material but remains in a mixed form. Illustrative methods to obtain extracts include expression of oil; absorption by steeping the plant material in a solvent such as, e.g., water or ethanol; maceration of the plant material; and distillation. In some cases two or more of these methods are combined, e.g., maceration and distillation. Other methods include, for instance, spagyric extraction, often involving fermentation, distillation and extraction of mineral components from the ash of a plant. In particularly useful embodiments according to the invention the extract is a composite that includes flavan-3-ols extracted from plant tissue by means of a solvent such as water or ethanol. The plant tissue that has been extracted may be from the roots, leaves, stems, flowers, fruits, seeds, components of any of those, or another part of a plant.

The term “grape seed extract” means an extract of grape seed(s). In particularly useful embodiments of the invention the grape seed extract is a commercially available product, such as is sold by, e.g., the company Naturex, SA.

The term “therapeutically effective amount” as used with respect to an ingredient or composition means an amount sufficient to elicit a desired biological response. The therapeutically effective amount or dose will depend on the age, sex and weight of the patient, and the current medical condition of the patient. The skilled artisan will be able to determine appropriate dosages depending on these and other factors in addition to the present disclosure. By definition, an amount of a vitamin or mineral adequate to correct or maintain basal levels of the vitamin or mineral above a depressed or deficient state are therapeutically effective. Non-limiting examples of therapeutically effective amounts for the present invention include the 2013 dietary reference intakes based on dietary equivalents (“Des”) for vitamin D compounds, cobalamin compounds (2.4 μg/day), and carotenoids such as vitamin A. For purposes of the present invention therapeutically effective amounts may include any larger amount up to the maximum level of daily nutrient intake that is likely to pose no risk of adverse effects. The dietary reference intakes (DRI) for those, also known as recommended dietary allowances (“RDA”), are published by Food and Nutrition Board of the Institute of Medicine, National Academy of Sciences.

The term “pharmaceutically acceptable” as used with respect to salts and complexes of compounds herein, means those which are useful in preparing a pharmaceutical composition that is generally safe, non-toxic and neither biologically nor otherwise undesirable and includes both those which are acceptable for veterinary use as well as those which are acceptable for human pharmaceutical use. Pharmaceutically acceptable salts are for example acid addition salts and basic salts but also include inner salts such as zwitterions. Acid addition salts are, e.g., salts made by addition of an acid, such as by HCl, HBr, H₂SO₄, H₃PO₄ or another inorganic acid or by citric acid or another organic acid. Basic salts are, e.g., salts having a cation selected from alkali or alkaline metals, e.g., Na⁺, K⁺, or Ca²⁺, or an ammonium ion N⁺(R1)(R2)(R3)(R4), wherein R1 to R4 independently of each other mean: hydrogen, an optionally substituted C1-C6 alkyl group, an optionally substituted C2-C6 alkenyl group, an optionally substituted C6-C10 aryl group, or an optionally substituted C6-C10 heteroaryl group. Further examples of pharmaceutically acceptable salts are described in REMINGTON'S PHARMACEUTICAL SCIENCES, 18 ed. (1990) and in ENCYCLOPEDIA OF PHARMACEUTICAL TECHNOLOGY.

The terms and abbreviations “milligram” and “mg” are equivalent. The terms and abbreviations “microgram” and “mcg” and “μg” are equivalent. And terms of measurement conform to their respective meanings in the International System of Units, also known as the Système international d'unités.

The abbreviation “I.U.” as used with respect to amounts of an ingredient means International Unit(s) as they are stated in the art of pharmacology for biologically active quantities of vitamins, hormones, some medications, vaccines, blood products, and similar biologically active substances.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

### Unit Dose Compositions

It has been has discovered that combining antioxidants from four particular classes provides surprisingly good benefits against prediabetes and its symptoms. Although vitamin C and vitamin E are commonly provided in prior art antioxidant supplements, the present invention does not require them nor does either of those vitamins fall into one of the four classes herein. The discovery further finds that providing at least one antioxidant that can also serve as a pro-oxidant is beneficial. Without being bound by the mechanism it is theorized that prediabetes is associated with increased damage to biochemical intermediates and tissues from specific types and ratios of free radicals, and that the present invention addresses their relative reactivity and prevalence. Moreover the varieties of compounds taught for use here will distribute to different tissues and cellular components, and thus without being bound by theory are believed to address the damage in each of those places that represent a signature pattern of prediabetes and diabetes.

CATEGORY I: Heteroatom-Based Natural Antioxidant Compounds.

These fall within a generic structural family having a short (2-5 carbons) alkyl chain with at one end a simple nitrogen group such as an unsubstituted amine or tri-methyl ammonium group, and at the neighboring carbon optionally an −0-acetyl or carboxyl group, and at the other end a group from an acid or acid derivative, such as a sulfonic, carboxylic or amide group, optionally augmented by an acid and or diimidazole group. The inventor theorizes without being so limited that radical oxidizing species in the body abstract a H atom from the alpha-carbon for the terminal amine of the antioxidant, and that the nitrogen atom is able to donate electron density (or in the case of the ammonium species, the nitrogen atom is able to donate electron density after a demethylation) to stabilize the adjacent radical. The radical may be further stabilized by donation from an acetyl ester oxygen at the same site. Or a captodative (“push-pull”) effect may stabilize the radical where an amine donates electron density from one side of the unpaired electron and a carboxyl center draws electron density from the other. Some molecules may have additional sited for stabilized abstraction of a hydrogen atom. This hypothesis assumes that the stabilized radical is less aggressively reactive and thus can be rehydrogenated or metabolized without perpetuating the more destructive branches of a reactive radical manifold. Again, the invention is not limited by this hypothetical mechanism.

In general it appears that it is most helpful to include a plurality of antioxidant compounds from this category, and the invention finds that it is useful to include as many as five or more. Preferred compounds include taurine, beta-alanine, acetyl-carnitine, agmatine, carnosine, cysteine, sulfinoalanine, hypotaurine, carnitine, lysine, anserine, histidine, arginine and ornithine. Particularly preferred compounds are taurine, carnosine, carnitine, acetylcarnitine, and agmatine, depicted in Table I below. However the invention is not so limited. Natural compounds, and particularly compounds having the stereoisomer found in the body are preferred, because they are safer in some cases (e.g., L-carnitine is less toxic than D-carnitine) and have an inherent advantage for docking with and being transported by or through natural stereoisomers of lipids, proteins, carbohydrates and nucleic acids. However the invention is not so limited.

In particular embodiments the composition is provided in a dose form. In certain embodiments the dose form includes taurine provided in a range selected from: 40 to 360 mg; 60 to 340 mg; 80 to 320 mg; 100 to 300 mg; 120 to 280 mg; 140 to 260 mg; 160 to 240 mg; 180 to 220 mg; or about 200 mg. In some embodiments the dose form includes beta-alanine provided in a range selected from: 10 to 90 mg; 15 to 85 mg; 20 to 80 mg; 25 to 75 mg; 30 to 70 mg; 35 to 65 mg; 40 to 60 mg; 45 to 55 mg; or about 50 mg. In various embodiments the dose form includes acetyl-L-carnitine provided in a range selected from: 20 to 180 mg; 30 to 170 mg; to 160 mg; 50 to 150 mg; 60 to 140 mg; 70 to 130 mg; 80 to 120 mg; 90 to 110 mg; or about 100 mg. In particular embodiments the dose form includes agmatine provided in a range selected from: 25 to 225 mg; 40 to 210 mg; 55 to 195 mg; 70 to 180 mg; 85 to 165 mg; 100 to 150 mg; 115 to 135 mg; or about 125 mg. In further embodiments the dose form provides L-carnosine provided in a range selected from: 5 to 45 mg; 7.5 to 42.5 mg; 10 to 40 mg; 12.5 to 37.5 mg; 15 to 35 mg; 17.5 to 32.5 mg; 20 to 30 mg; 22.5 to 27.5 mg; or about 25 mg. In some embodiments the dose form provides an 8:2:4:5:1 respective ratio of taurine, beta-alanine, acetyl-L-carnitine, agmatine, and L-carnosine.

TABLE I
ANTIOXIDANTS WITH A STABILIZING HETEROATOM
Taurine

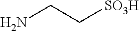

Beta-alanine

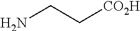

Agmatine

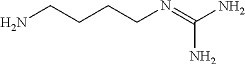

Acetyl-L-carnitine

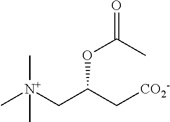

L-carnosine

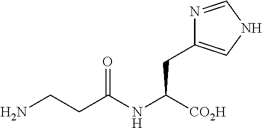 [SMILES: NCCC(=O)N[C@@H](Cc1c[nH]cn1)C(=O)O]

CATEGORY II: Natural Compounds Having a Conjugated Segment.

Conjugation is the alternation of double and single bonds, and particularly where several double bonds are involved in the sequence, this provides a stabilized site for hydrogen abstraction at saturated carbon atoms that are adjacent to a double bond in the series, whether at the terminus of a conjugated segment, or at a branch along the chain. This is true even when the branch is as short as a methyl group.

The inventor has found that vitamin D compounds and cobalamin compounds are useful of this purpose, and that including a representative antioxidant compound from each of those categories is particularly useful. In certain embodiments at least one carotenoid is also included.

Useful vitamin D compounds for purposes of the invention include: 7-dehydrocholesterol, also known as previtamin-D₃; vitamin D₂, also known as ergocholecalciferol; vitamin D₃, also known as cholecalciferol and activated 7-dehydrocholesterol; 25-D, also known as 25-hydroxyvitamin D, calcidiol and calcifediol; and 1,25-D, also known as 1,25-dihydroxycholecalciferol, 1,25-dihydroxyvitamin D₃ and calcitriol. In particularly preferred embodiments the vitamin D compound is vitamin D₃, depicted in Table II below, but the invention is not so limited. In particular embodiments the composition is provided in a dose form comprising vitamin D₃ provided in a range selected from: 500 to 4,500 I.U.; 750 to 4,250 I.U.; 1,000 to 4,000 I.U.; 1,250 to 3,750 I.U.; 1,500 to 3,500 I.U.; 1,750 to 3,250 I.U.; 2,000 to 3,000 I.U.; 2,250 to 2,750 I.U.; or about 2,500 I.U.

Preferred cobalamin compounds for the invention include: methylcobalamin; hydroxocobalamin; cyanocobalamin; and adenosylcobalamin. Among other properties cobalamin compounds can bind the radical NO·. Methylcobalamin is particularly preferred, and is depicted in Table II below, but the invention is not so limited. In some embodiments the composition is provided in a dose form comprising a cobalamin compound provided in a range selected from: 20 to 220 μg; 30 to 210 μg; 40 to 200 μg; 50 to 190 μg; 60 to 180 μg; 70 to 170 μg; 80 to 160 μg; 90 to 150 μg; 100 to 140 μg; 110 to 130 μg; or about 120 μg.

Preferred carotenoids for purposes of the invention include the lettered carotenes, lycopene, neurosporene, phytofluene, phytoene, xanthophyll, canthaxanthin, cryptoxanthin, zeaxanthin, astaxanthin, lutein, and rubixanthin. A particularly preferred carotenoid is lutein, depicted in Table II below, but the invention is not so limited. In certain embodiments the composition is provided in a dose form comprising lutein in a range selected from: 1 to 9 mg; 1.5 to 8.5 mg; 2 to 8 mg; 2.5 to 7.5 mg; 3 to 7 mg; 3.5 to 6.5 mg; 4 to 6 mg; 4.5 to 5.5 mg; or about 5 mg.

In certain embodiments the composition is provided in a dose form that comprises per each milligram of carotenoid compound: 500 I.U. vitamin D compound and 24 μg cobalamin compound.

TABLE II
ANTIOXIDANTS WITH CONJUGATED SEGMENTS
Vitamin D3

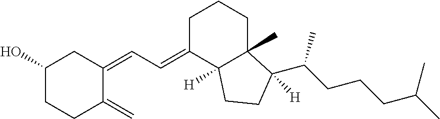 [SMILES: C=C1CC[C@H](O)C/C1=C/C=C1\CCC[C@]2(C)[C@@H]([C@H](C)CCCC(C)C)CC[C@@H]12]

Methylcobalamin

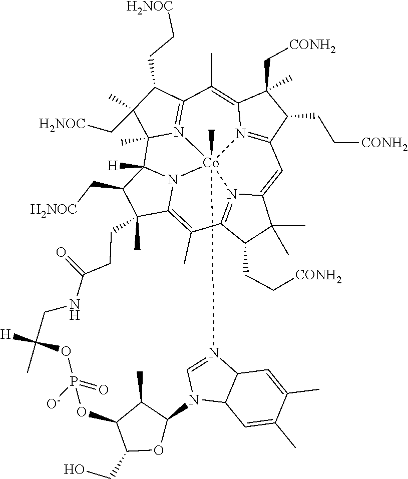

Lutein

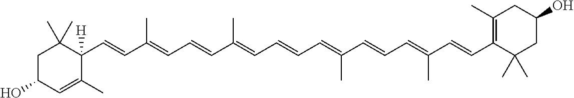 [SMILES: CC1=C[C@H](O)CC(C)(C)[C@H]1/C=C/C(C)=C/C=C/C(C)=C/C=C/C=C(C)/C=C/C=C(C)/C=C/C1=C(C)C[C@@H](O)CC1(C)C]

CATEGORY III: Compounds that have a Disulfide Bond.

Aggressive radicals can attack disulfide bonds, such as by abstracting an electron from one of the sulfurs, or by forming a bond with one of the S atoms while breaking the S—S bond and leaving the other S atom as a radical —S that is stabilized by the electron-rich environment of the sulfur atom itself.

Particularly useful disulfides include: natural products and especially botanical natural products; derivatives of B-vitamins; and lipoic acid and its derivatives. Certain preferred disulfide antioxidants are natural products, including: lipoic acid, glutathione disulfide, and L-cystine, each of which is common in mammalian metabolism; asparagusic acid, commonly found in asparagus plants; lenthionine, commonly found in Shiitake mushrooms; and disulfide compounds from the Allium (onion and garlic) family, including ajoene, allithiamine, allyl propyl disulfide, and diallyl disulfide. Certain other preferred disulfide antioxidants are derivatives of small-molecule B vitamins, including fursultiamine (for B₁), pantethine and its salts (for B₅). prosultiamine (for B₁), pyritinol (for B₆), and sulbutiamine (for B₁), each of which is depicted in Table III below. Particularly preferred disulfide antioxidants for purposes of the invention are lipoic acid or its derivative lipoamide, but the invention is not so limited.

In certain embodiments the composition is provided in a dose form comprising alpha-lipoic acid provided in a range selected from: 100 to 900 μg; 150 to 850 μg; 200 to 800 μg; 250 to 750 μg; 300 to 700 μg; 350 to 650 μg; 400 to 600 μg; 450 to 550 μg; or about 500 μg.

TABLE III
DISULFIDES
Lipoic Acid

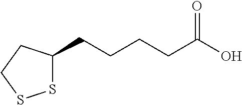 [SMILES: O=C(O)CCCC[C@@H]1CCSS1]

Lipoamide

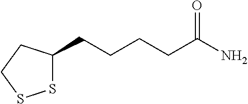 [SMILES: NC(=O)CCCC[C@@H]1CCSS1]

Asparagusic acid

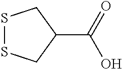

Diallyl disulfide

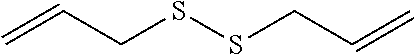

Allyl propyl sulfide

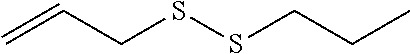

Ajoene

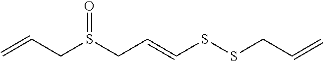

Lenthionine

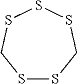

Pyritinol

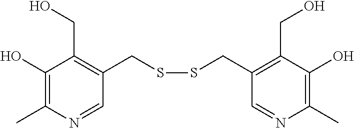 [SMILES: Cc1ncc(CSSCc2cnc(C)c(O)c2CO)c(CO)c1O]

Cystine

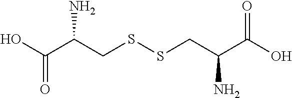 [SMILES: N[C@H](CSSC[C@H](N)C(=O)O)C(=O)O]

Allithiamine

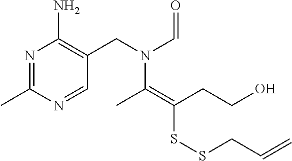 [SMILES: C=CCSS/C(CCO)=C(\C)N(C=O)Cc1cnc(C)nc1N]

Fursultiamine

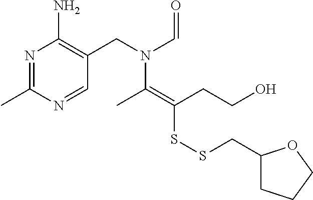 [SMILES: C/C(=C(/CCO)SSCC1CCCO1)N(C=O)Cc1cnc(C)nc1N]

Prosultiamine

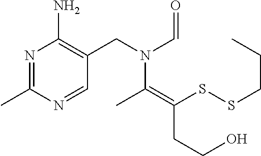 [SMILES: CCCSS/C(CCO)=C(/C)N(C=O)Cc1cnc(C)nc1N]

Sulbutiamine

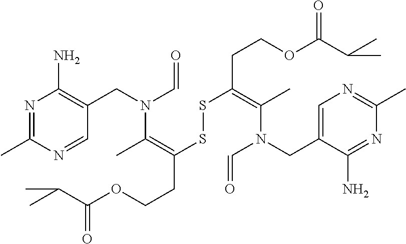

Pantethine

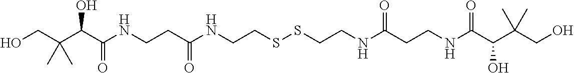 [SMILES: CC(C)(CO)[C@H](O)C(=O)NCCC(=O)NCCSSCCNC(=O)CCNC(=O)[C@H](O)C(C)(C)CO]

Glutathione disulfide

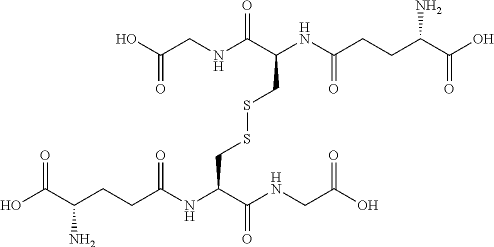 [SMILES: N[C@@H](CCC(=O)N[C@@H](CSSC[C@H](NC(=O)CC[C@H](N)C(=O)O)C(=O)NCC(=O)O)C(=O)NCC(=O)O)C(=O)O]

CATEGORY IV: Flavan-3-ol Compounds.

Flavanol-3-ol compounds are flavonoids with numerous sites for abstraction of a phenolic hydrogen, to leave an aromatically delocalized and thus stabilized oxyradical —O·.

Preferred flavan-3-ols for purposes of the invention are depicted in Table IV below: catechin (C), epicatechin (E), gallate (G), epigallocatechin (EGC), epigallocatechin gallate (EGCG), theaflavins, theaflavin-3-gallate, and thearubigins. Molecular variants based on stereoisomers, glycosides, oligomers and polymers are also preferred, in particular proanthocyanidins and their subset the procyanidins. Particularly preferred flavan-3-ols are (+)-catechin, (+)-gallocatechin, (−)-epicatechin, (−)-epigallocatechin, (−)-epigallocatechin gallate (EGCG), (−)-epicatechin 3-gallate, and procyanidins and other proanthocyanidins.

In certain preferred embodiments the flavan-3-ols are provided in the form of a mixture found in nature, such as in grape seed extract. Grape seed extract comprises phenolic antioxidants, such as for instance, epicatechin and oligomers of epicatechin. However the invention is not so limited. Other desirable sources of mixed flavan-3ols include, for example, apples (especially Red Delicious and Granny Smith varieties), maritime pine bark, cinnamon, aronia fruit, cocoa beans, grape skins, red wine, bilberry, cranberry, black currant, green tea, black tea, oak heartwood (Quercus petraea and Q. robur), açai oil, field beans (Vicia faba), and Gallipoli rose. In various embodiments the composition is provided in a dose form that comprises grape seed extract in a range selected from: 10-90 mg; 15 to 85 mg; 20 to 80 mg; 25 to 75 mg; 30 to 70 mg; 35 to 65 mg; 40 to 60 mg; 45 to 55 mg; or about 50 mg.

TABLE IV
ILLUSTRATIVE FLAVAN-3-OLS
(+)-Catechin, C,
[Oligomers = Procyanidins]

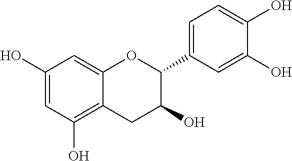 [SMILES: Oc1cc(O)c2c(c1)O[C@H](c1ccc(O)c(O)c1)[C@@H](O)C2]

(−)-Epicatechin (cis), EC
[Oligomers = Procyanidins]

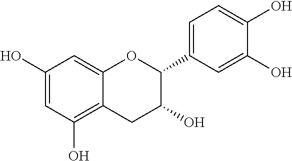 [SMILES: Oc1cc(O)c2c(c1)O[C@H](c1ccc(O)c(O)c1)[C@H](O)C2]

(−)-Epigallocatechin, EGC
[Oligomers = Prodelphinidins]

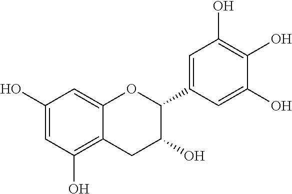 [SMILES: Oc1cc(O)c2c(c1)O[C@H](c1cc(O)c(O)c(O)c1)[C@H](O)C2]

(−)-Epicatechin gallate, ECG

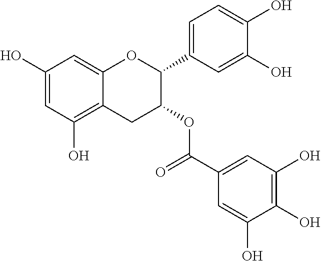 [SMILES: O=C(O[C@@H]1Cc2c(O)cc(O)cc2O[C@@H]1c1ccc(O)c(O)c1)c1cc(O)c(O)c(O)c1]

(−)-Epigallocatechin gallate, EGCG,

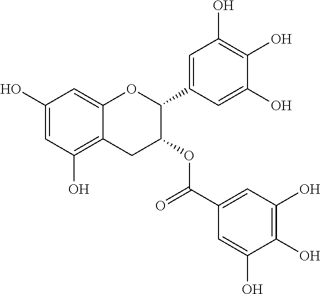 [SMILES: O=C(O[C@@H]1Cc2c(O)cc(O)cc2O[C@@H]1c1cc(O)c(O)c(O)c1)c1cc(O)c(O)c(O)c1]

Epiafzelechin

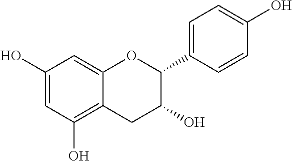 [SMILES: Oc1ccc([C@H]2Oc3cc(O)cc(O)c3C[C@H]2O)cc1]

Fisetinidol

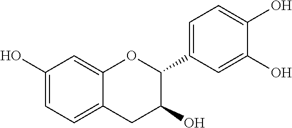 [SMILES: Oc1ccc2c(c1)O[C@H](c1ccc(O)c(O)c1)[C@@H](O)C2]

Mesquitol

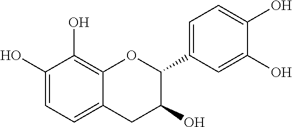 [SMILES: Oc1ccc([C@H]2Oc3c(ccc(O)c3O)C[C@@H]2O)cc1O]

Guibourtinidol
[Oligomers = Proguibourtinidins]

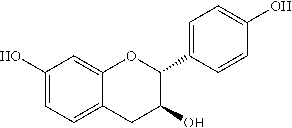 [SMILES: Oc1ccc([C@H]2Oc3cc(O)ccc3C[C@@H]2O)cc1]

Robinetinidol
[Oligomers = Prorobinetinidins]

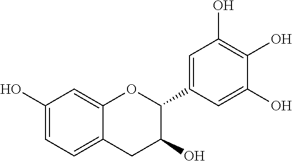 [SMILES: Oc1ccc2c(c1)O[C@H](c1cc(O)c(O)c(O)c1)[C@@H](O)C2]

Antioxidants that are Also Pro-Oxidants:

Without being bound by theory it is believed that the pro-oxidant properties of some antioxidants employed in the present invention contribute to establishing an oxidative balance in the tissues, and to maintaining an equilibrium in the oxidation states of the metals that are active there. It is also conceivable that this may help to expose and flush out latent populations of deleteriously oxidizing species found there. Among other observations, antioxidants that exert a pro-oxidant effect in the liver are particularly useful. This may possibly arise from a readier flushing of some metals from that tissue when they are in an alternative oxidation state. Comments follow on the pro-oxidant capabilities of the antioxidant classes for which use is taught here. Note that some members of an antioxidant class may be pro-oxidants while others may not be.

Most compounds with a stabilizing heteroatom do not appear to be pro-oxidants. Their role in up- and down-regulation of biochemical pathways in some cases can alter the oxidative stress. However for purposes of the invention that alone does not make them pro-oxidants. The pro-oxidant properties of vitamin D in native tissues are not clear, though it has pro-oxidative effects in breast cancer cells. Cobalamin compounds do not appear to be pro-oxidants, nor do most carotenoids.

Antioxidants with disulfide bonds may be pro-oxidants, for instance alpha-lipoic acid is.

Flavan-3-ols behave somewhat analogously to ascorbate and commonly have prooxidant activity even at low concentrations (unlike the sterically protected phenolics such as the synthetic antioxidants BHA and BHT); this is evident for instance by their initiation of lipid peroxidation in the presence of O₂ and a metal such as Cu, Al, Zn, Ca, Mg or Cd.

Relative Ratios in the Compositions.

In certain embodiments the composition is provided in a dose form in which the total weight of compounds from each of Categories, I, II, III and IV is provided in a respective ratio of 1,000:10:100:1, or wherein each number in the ratio may be varied by ±80% independently of the others.

Pharmaceutical Formulations

The unit dose compositions described herein comprises a mixture of the antioxidants described above alone or in combination with one or more pharmaceutically-acceptable carriers. In one aspect, the unit dose compositions are an intimate blend of the antioxidants. In one aspect, the antioxidants described herein can be admixed with one another in dry form or in a solution to ensure the components are homogeneously mixed with one another.

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous).

As used herein, “unit dose,” which is also referred to as “dose,” “dosage,” or “dosage form” can refer to physically discrete units suitable for use in a subject, each unit containing a predetermined quantity of a disclosed compound and/or a pharmaceutical composition thereof calculated to produce the desired response or responses in association with its administration. Thus, the pharmaceutical compositions of the present disclosure can be presented as discrete units suitable for oral administration such as, for example, tablets, capsules, caplets, gelcaps, geltabs, pills, lozenges, chewable articles (e.g., a gummy), dissolvable strips (e.g., placement under the tongue), or any other suitable form for oral administration each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethyl carbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like.

Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

In one aspect, the unit dose composition is a capsule. The capsule can include one or more flow agents useful in the manufacturing of the capsule. The flow agent can ensure the capsule is filled with the proper and consistent amount of the antioxidants described herein. The flow agent can also absorb moisture and prevent the formulation from forming lumps that would result in capsule inconsistencies as well as promote quick encapsulation of the antioxidants.

Examples of flow agents include, but are not limited to, magnesium stearate, microcrystalline cellulose (MCC), di-calcium phosphate, silica dioxide, or any combination thereof.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik and angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

### Aspects

Aspect 1. A unit dose composition comprising the following antioxidant compounds:
a) a plurality of heteroatom-containing natural antioxidant compounds having the formula X—CH(R¹)—CH(R²)—(CH₂)ₘ—Z, wherein:
   i) X is one of the following moieties or a pharmaceutically acceptable salt thereof: —NH₂ or —N(CH₃)₃;
   ii) R¹ is —H, —CO₂H, or a pharmaceutically acceptable salt of —CO₂H;
   iii) R² is —H, —OH, or —O-acetyl;
   iv) m is 0, 1, 2, or 3; and
   v) Z is one of the following moieties or a pharmaceutically acceptable salt thereof: —SH, —SO₂H, —SO₃H, —CO₂H, —N═C(—NH₂)—NH₂, —CH(—NH₂)—CO₂H; -imidazolyl; or —C(═O)—NH—CH(—CO₂H)—CH₂—Y where Y is 4-imidazolyl or 1-methyl-4-imidazolyl;
b) a plurality of antioxidant compounds having a conjugated segment, comprising at least one vitamin D compound and at least one cobalamin compound;
c) at least one of the antioxidant compounds comprising a disulfide bond; and
d) at least one of the antioxidant compounds is a flavan-3-ol compound,
wherein the unit dose composition is for oral administration.
   Aspect 2. The unit dose composition of Aspect 1 wherein the compounds having the formula X—CH₂—CH(R)—(CH₂)ₘ—Z comprise a plurality selected from the group consisting of the following compounds and their pharmaceutically acceptable salts: taurine, beta-alanine, acetyl-L-carnitine, agmatine, carnosine, cysteine, sulfinoalanine, hypotaurine, carnitine, lysine, anserine, histidine, arginine, and ornithine.
   Aspect 3. The unit dose composition of Aspect 1 wherein the compounds having the formula X—CH₂—CH(R)—(CH₂)ₘ—Z comprise at least each of taurine, beta-alanine, acetyl-L-carnitine, agmatine, and L-carnosine.
   Aspect 4. The unit dose composition of Aspect 1 wherein the plurality of antioxidant compounds having a conjugated segment comprises at least one of vitamin D₃ and methylcobalamin.
   Aspect 5. The unit dose composition of Aspect 1 wherein the plurality of antioxidant compounds having a conjugated segment further comprise a carotenoid selected from the group consisting of lettered carotenes, lycopene, neurosporene, phytofluene, phytoene, canthaxanthin, cryptoxanthin, zeaxanthin, astaxanthin, lutein, and rubixanthin.
   Aspect 6. The unit dose composition of Aspect 1 wherein the at least one antioxidant compound comprising a disulfide bond is selected from the group consisting of alpha-lipoic acid, lipoamide, glutathione disulfide, L-cystine, asparagusic acid, lenthionine, ajoene, allithiamine, allyl propyl disulfide, diallyl disulfide, fursultiamine, pantethine and its salts, prosultiamine, pyritinol, and sulbutiamine.
   Aspect 7. The unit dose composition of Aspect 1 wherein the at least one antioxidant compound comprising a disulfide bond comprises alpha-lipoic acid.
   Aspect 8. The unit dose composition of Aspect 1 wherein the at least one flavan-3-ol compound is selected from the group consisting of monomers, oligomers, polymers, glycosides and pharmaceutically acceptable salts of the following: (+)-catechin, (−)-epicatechin, epigallocatechin, epicatechin gallate, epigallocatechin gallate, epiafzelechin, and fisetinidol.
   Aspect 9. The unit dose composition of Aspect 1 wherein the at least one flavan-3-ol compound is provided in the form of grape seed extract.
   Aspect 10. The unit dose composition of Aspect 1 wherein at least one of a disulfide compound or a flavan-3-ol is both an antioxidant and a pro-oxidant.
   Aspect 11. The unit dose composition of Aspect 1 wherein the composition comprises:
a) taurine;
b) beta-alanine;
c) acetyl-L-carnosine;
d) agmatine;
e) L-carnosine;
f) vitamin D₃;
g) methylcobalamin;
h) lutein;
i) grape seed extract; and
j) alpha-lipoic acid.
   Aspect 12. The unit dose composition of Aspect 1 wherein:
a) compounds having the formula X—CH₂—CH(R)—(CH₂)ₘ—Z comprise at least:
   i) taurine in an amount selected from the range of 40 to 360 mg;
   ii) beta-alanine in an amount selected from the range of 10 to 90 mg;
   iii) acetyl-L-carnosine in an amount selected from the range of 20 to 180 mg;
   iv) agmatine in an amount selected from the range of 25 to 225 mg; and
   v) L-carnosine in an amount selected from the range of 5 to 45 mg;
b) compounds having a conjugated segment comprise at least:
   vi) vitamin D₃ in an amount selected from the range of 500 to 4,500 I.U.; and
   vii) methylcobalamin in an amount selected from the range of 20 to 220 μg; and
c) a plurality of flavan-3-ols is provided in the form of grape seed extract in an amount selected from the range of 10 to 90 mg.
   Aspect 13. The unit dose composition of Aspect 12 wherein the composition further comprises:
e) a carotenoid, lutein, in an amount selected from the range of 1 to 9 mg; and
f) a disulfide compound, alpha-lipoic acid, in an amount selected from the range of 100 to 900 μg.
   Aspect 14. The unit dose composition of Aspect 1 wherein the composition comprises:
a) taurine in an amount of 200 mg;
b) beta-alanine in an amount of 50 mg;
c) acetyl-L-carnosine in an amount of 100 mg;
d) agmatine in an amount selected of 125 mg;
e) L-carnosine in an amount of 25 mg;
f) vitamin D₃ in an amount of 2,500 I.U.;
g) methylcobalamin in an amount of 120 μg;
h) lutein in an amount of 5 mg;
i) grape seed extract in an amount of 50 mg; and
j) alpha-lipoic acid in an amount of 500 μg.
   Aspect 5. The unit dose composition of Aspect 1 wherein the composition comprises:
a) a plurality of heteroatom-containing natural antioxidant compounds having the formula X—CH(R¹)—CH(R²)—(CH₂)ₘ—Z, wherein:
   i) X is one of the following moieties or a pharmaceutically acceptable salt thereof: —NH₂ or —N(CH₃)₃;
   ii) R¹ is —H, —CO₂H, or a pharmaceutically acceptable salt of —CO₂H;
   iii) R² is —H, —OH, or —O-acetyl;
   iv) m is 0, 1, 2, or 3; and
   v) Z is one of the following moieties or a pharmaceutically acceptable salt thereof: —SH, —SO₂H, —SO₃H, —CO₂H, —N═C(—NH₂)—NH₂, —CH(—NH₂)—CO₂H; -imidazolyl; or —C(═O)—NH—CH(—CO₂H)—CH₂—Y where Y is 4-imidazolyl or 1-methyl-4-imidazolyl;
wherein the compounds having the formula X—CH₂—CH(R)—(CH₂)ₘ—Z comprise a plurality selected from the group consisting of the following compounds and their pharmaceutically acceptable salts: taurine, beta-alanine, acetyl-L-carnitine, agmatine, carnosine, cysteine, sulfinoalanine, hypotaurine, carnitine, lysine, anserine, histidine, arginine, and ornithine;
b) a plurality of antioxidant compounds having a conjugated segment, comprising at least one vitamin D compound in an amount selected from a range of 500 to 4,500 I.U., and at least one cobalamin compound in an amount selected from a range of 20 to 220 and at least one carotenoid selected from the group consisting of lettered carotenes, lycopene, neurosporene, phytofluene, phytoene, canthaxanthin, cryptoxanthin, zeaxanthin, astaxanthin, lutein, and rubixanthin;
c) at least one of the antioxidant compounds comprises a disulfide bond and is selected from the group consisting of alpha-lipoic acid, lipoamide, glutathione disulfide, L-cystine, asparagusic acid, lenthionine, ajoene, allithiamine, allyl propyl disulfide, diallyl disulfide, fursultiamine, pantethine and its salts. prosultiamine, pyritinol, and sulbutiamine; and
d) at least one of the antioxidant compounds is a flavan-3-ol compound that is selected from the group consisting of monomers, oligomers, polymers, glycosides and pharmaceutically acceptable salts of the following: (+)-catechin, (−)-epicatechin, epigallocatechin, epicatechin gallate, epigallocatechin gallate, epiafzelechin, and fisetinidol;
wherein at least one antioxidant compound in the composition is also a pro-oxidant; and
wherein the composition is provided in a dose form in which the total weight of compounds shown for each of (a), (b), (c), and (d) above is provided in a respective ratio of 1,000:10:100:1, yet wherein each number in the ratio may be varied by +/−80% independently of the others.
   Aspect 16. The unit dose composition of Aspect 1 wherein the unit dose composition is a tablet, capsule, caplet, gelcap, geltab, pill, lozenge, chewable article, or dissolvable strip.
   Aspect 17. The unit dose composition of Aspect 1 wherein the unit dose composition is a capsule.
   Aspect 18. The unit dose composition of Aspect 1 wherein the unit dose composition is a capsule, and the capsule further comprises a flow agent.
   Aspect 19. A method for treatment of prediabetes in a subject comprising administering the unit dose composition of Aspect 1 to the subject.

The embodiments of the invention as described herein are merely illustrative and are not exclusive. Numerous additions, variations, derivations, permutations, equivalents, combinations and modifications of the above-described invention will be apparent to persons of ordinary skill in the relevant arts and are within the scope and spirit of the invention. The invention as described herein contemplates the use of those alternative embodiments without limitation.

1) A unit dose composition comprising the following antioxidant compounds:
a) a plurality of heteroatom-containing natural antioxidant compounds having the formula X—CH(R¹)—CH(R²)—(CH₂)ₘ—Z, wherein:
i) X is one of the following moieties or a pharmaceutically acceptable salt thereof: —NH₂ or —N(CH₃)₃;
ii) R¹ is —H, —CO₂H, or a pharmaceutically acceptable salt of —CO₂H;
iii) R² is —H, —OH, or −O-acetyl;
iv) m is 0, 1, 2, or 3; and
v) Z is one of the following moieties or a pharmaceutically acceptable salt thereof: —SH, —SO₂H, —SO₃H, —CO₂H, —N═C(—NH₂)—NH₂, —CH(—NH₂)—CO₂H; -imidazolyl; or —C(═O)—NH—CH(—CO₂H)—CH₂—Y where Y is 4-imidazolyl or 1-methyl-4-imidazolyl;
b) a plurality of antioxidant compounds having a conjugated segment, comprising at least one vitamin D compound and at least one cobalamin compound;
c) at least one of the antioxidant compounds comprising a disulfide bond; and
d) at least one of the antioxidant compounds is a flavan-3-ol compound,
wherein the unit dose composition is for oral administration.

2) The unit dose composition of claim 1 wherein the compounds having the formula X—CH₂—CH(R)—(CH₂)ₘ—Z comprise a plurality selected from the group consisting of the following compounds and their pharmaceutically acceptable salts: taurine, beta-alanine, acetyl-L-carnitine, agmatine, carnosine, cysteine, sulfinoalanine, hypotaurine, carnitine, lysine, anserine, histidine, arginine, and ornithine.

3) The unit dose composition of claim 1 wherein the compounds having the formula X—CH₂—CH(R)—(CH₂)ₘ—Z comprise at least each of taurine, beta-alanine, acetyl-L-carnitine, agmatine, and L-carnosine.

4) The unit dose composition of claim 1 wherein the plurality of antioxidant compounds having a conjugated segment comprises at least one of vitamin D₃ and methylcobalamin.

5) The unit dose composition of claim 1 wherein the plurality of antioxidant compounds having a conjugated segment further comprise a carotenoid selected from the group consisting of lettered carotenes, lycopene, neurosporene, phytofluene, phytoene, canthaxanthin, cryptoxanthin, zeaxanthin, astaxanthin, lutein, and rubixanthin.

6) The unit dose composition of claim 1 wherein the at least one antioxidant compound comprising a disulfide bond is selected from the group consisting of alpha-lipoic acid, lipoamide, glutathione disulfide, L-cystine, asparagusic acid, lenthionine, ajoene, allithiamine, allyl propyl disulfide, diallyl disulfide, fursultiamine, pantethine and its salts, prosultiamine, pyritinol, and sulbutiamine.

7) The unit dose composition of claim 1 wherein the at least one antioxidant compound comprising a disulfide bond comprises alpha-lipoic acid.

8) The unit dose composition of claim 1 wherein the at least one flavan-3-ol compound is selected from the group consisting of monomers, oligomers, polymers, glycosides and pharmaceutically acceptable salts of the following: (+)-catechin, (−)-epicatechin, epigallocatechin, epicatechin gallate, epigallocatechin gallate, epiafzelechin, and fisetinidol.

9) The unit dose composition of claim 1 wherein the at least one flavan-3-ol compound is provided in the form of grape seed extract.

10) The unit dose composition of claim 1 wherein at least one of a disulfide compound or a flavan-3-ol is both an antioxidant and a pro-oxidant.

11) The unit dose composition of claim 1 wherein the composition comprises:
a) taurine;
b) beta-alanine;
c) acetyl-L-carnosine;
d) agmatine;
e) L-carnosine;
f) vitamin D₃;
g) methylcobalamin;
h) lutein;
i) grape seed extract; and
j) alpha-lipoic acid.

12) The unit dose composition of claim 1 wherein:
a) compounds having the formula X—CH₂—CH(R)—(CH₂)ₘ—Z comprise at least:
i) taurine in an amount selected from the range of 40 to 360 mg;
ii) beta-alanine in an amount selected from the range of 10 to 90 mg;
iii) acetyl-L-carnosine in an amount selected from the range of 20 to 180 mg;
iv) agmatine in an amount selected from the range of 25 to 225 mg; and
v) L-carnosine in an amount selected from the range of 5 to 45 mg;
b) compounds having a conjugated segment comprise at least:
i) vitamin D₃ in an amount selected from the range of 500 to 4,500 I.U.; and
ii) methylcobalamin in an amount selected from the range of 20 to 220 μg; and
c) a plurality of flavan-3-ols is provided in the form of grape seed extract in an amount selected from the range of 10 to 90 mg.

13) The unit dose composition of claim 12 wherein the composition further comprises:
a) a carotenoid, lutein, in an amount selected from the range of 1 to 9 mg; and
b) a disulfide compound, alpha-lipoic acid, in an amount selected from the range of 100 to 900 μs.

14) The unit dose composition of claim 1 wherein the composition comprises:
a) taurine in an amount of 200 mg;
b) beta-alanine in an amount of 50 mg;
c) acetyl-L-carnosine in an amount of 100 mg;
d) agmatine in an amount selected of 125 mg;
e) L-carnosine in an amount of 25 mg;
f) vitamin D₃ in an amount of 2,500 I.U.;
g) methylcobalamin in an amount of 120 μg;
h) lutein in an amount of 5 mg;
i) grape seed extract in an amount of 50 mg; and
j) alpha-lipoic acid in an amount of 500 μg.

15) The unit dose composition of claim 1 wherein the composition comprises:
a) a plurality of heteroatom-containing natural antioxidant compounds having the formula X—CH(R¹)—CH(R²)—(CH₂)ₘ—Z, wherein:
i) X is one of the following moieties or a pharmaceutically acceptable salt thereof: —NH₂ or —N(CH₃)₃;
ii) R¹ is —H, —CO₂H, or a pharmaceutically acceptable salt of —CO₂H;
iii) R² is —H, —OH, or —O-acetyl;
iv) m is 0, 1, 2, or 3; and
v) Z is one of the following moieties or a pharmaceutically acceptable salt thereof: —SH, —SO₂H, —SO₃H, —CO₂H, —N═C(—NH₂)—NH₂, —CH(—NH₂)—CO₂H; -imidazolyl; or —C(═O)—NH—CH(—CO₂H)—CH₂—Y where Y is 4-imidazolyl or 1-methyl-4-imidazolyl;
wherein the compounds having the formula X—CH₂—CH(R)—(CH₂)ₘ—Z comprise a plurality selected from the group consisting of the following compounds and their pharmaceutically acceptable salts: taurine, beta-alanine, acetyl-L-carnitine, agmatine, carnosine, cysteine, sulfinoalanine, hypotaurine, carnitine, lysine, anserine, histidine, arginine, and ornithine;
b) a plurality of antioxidant compounds having a conjugated segment, comprising at least one vitamin D compound in an amount selected from a range of 500 to 4,500 I.U., at least one cobalamin compound in an amount selected from a range of 20 to 220 and at least one carotenoid selected from the group consisting of lettered carotenes, lycopene, neurosporene, phytofluene, phytoene, canthaxanthin, cryptoxanthin, zeaxanthin, astaxanthin, lutein, and rubixanthin;
c) at least one of the antioxidant compounds comprises a disulfide bond and is selected from the group consisting of alpha-lipoic acid, lipoamide, glutathione disulfide, L-cystine, asparagusic acid, lenthionine, ajoene, allithiamine, allyl propyl disulfide, diallyl disulfide, fursultiamine, pantethine and its salts. prosultiamine, pyritinol, and sulbutiamine; and
d) at least one of the antioxidant compounds is a flavan-3-ol compound that is selected from the group consisting of monomers, oligomers, polymers, glycosides and pharmaceutically acceptable salts of the following: (+)-catechin, (−)-epicatechin, epigallocatechin, epicatechin gallate, epigallocatechin gallate, epiafzelechin, and fisetinidol;
wherein at least one antioxidant compound in the composition is also a pro-oxidant; and
wherein the composition is provided in a dose form in which the total weight of compounds shown for each of (a), (b), (c), and (d) above is provided in a respective ratio of 1,000:10:100:1, yet wherein each number in the ratio may be varied by +/−80% independently of the others.

16) The unit dose composition of claim 1 wherein the unit dose composition is a tablet, capsule, caplet, gelcap, geltab, pill, lozenge, chewable article, or dissolvable strip.

17) The unit dose composition of claim 1 wherein the unit dose composition is a capsule.

18) The unit dose composition of claim 1 wherein the unit dose composition is a capsule, and the capsule further comprises a flow agent.

19) A method for treatment of prediabetes in a subject comprising administering the unit dose composition of claim 1 to the subject.
